(19) United States
(12) Patent Application Publication
(10) Pub. No.: US 2023/0355172 A1
(43) Pub. Date: Nov. 9, 2023

(54) TISSUE-PROSTHESIS INTERFACE SENSOR SYSTEM

(71) Applicant: Scinetics, Inc., Abingdon, MD (US)

(72) Inventor: Jeffrey L. Gair, JR., Abingdon, MD (US)

(73) Assignee: Scinetics, Inc., Abingdon, MD (US)

(21) Appl. No.: 18/142,694

(22) Filed: May 3, 2023

Related U.S. Application Data
(60) Provisional application No. 63/338,091, filed on May 4, 2022.

(51) Int. Cl. A61B 5/00; A61F 2/70

(52) U.S. Cl. CPC A61B 5/4851; A61B 5/6811; A61F 2/70; A61F 2002/704; A61F 2002/7615

(57) ABSTRACT

A tissue-prosthesis interface sensor system used to measure pressure values exerted across the surface of the limb. The system provides a high-speed method of measuring pressure values through sensors located on the surface of a wearer's skin.

### FIELD OF THE INVENTION

The disclosure of the present patent invention relates to orthotics, exoskeletons, prosthetics, and prosthesis systems.

### BACKGROUND

Prosthesis systems (or prosthetic devices) connect a residual limb of a user to a prosthetic limb. A typical prosthesis system consists of a custom fitted socket that enwraps the residual limb, the prosthesis or artificial limb, cuffs and belts that attach it to the body, and/or prosthetic liners that cushion the area which contacts the skin. Prosthesis systems and specifically the sockets are not mass-produced and sold in stores. Instead, like dentures or eyeglasses, prosthetic devices are prescribed by a clinical practitioner after consultation with the patient, a prosthetist, and a physical therapist. Sockets must be custom made to securely fit the residual limb of a patient. Sockets generally must be fit by a clinical prosthetist. A prosthetist requires extensive training to learn how to mold, align, and fit a socket to an individual's limb.

Current prosthetic sockets require a complex and labor-intensive manufacturing process. A prosthetist must take several measurements of the residual limb to create a plaster cast of the residual limb. From there, a thermoplastic sheet is heated and vacuum-formed around the plaster mold to form a test socket. The prosthetist works with the patient to ensure the test socket fits, makes required modifications, and then creates a permanent socket. Optimal socket fit between the residual limb and socket is critical because it impacts the functionality and usability of the prosthesis system. If the socket is too loose, it can reduce areas of contact and create pockets between the residual limb and socket or liner. Pockets can accumulate sweat which damages the skin and results in rashes. If the socket is too tight, it increases contact pressure on the skin causing the skin to break down over time.

Current prosthesis systems are unable to easily adjust to changes in an individual's limb size. Limb size may change if the patient gains or loses weight, or may change as the patient grows in the case of a young patient. If the limb does not fit within the socket properly, it can render the prosthetic device less effective, ineffective, or even hazardous if it becomes unstable, for example, while the user is driving or walking. Consequently, patients may have to see a prosthetist to remold, align, and fit a new socket which comes at a significant cost.

Prosthetic limbs require skills that a patient must learn over time through extensive physical therapy. One issue patients face is the inability to feel sensation in the prostheses. Another issue is that patients experience phantom limb syndrome where the patient feels as if the amputated limb is still there. Consequently, performing even menial tasks requires active learning where the skill or task is explicitly practiced through a training regimen. Prosthesis embodiment refers to the ability of a user to use a prosthesis as if it belonged to the body. Studies reveal that prosthesis embodiment is more likely to occur when the user engages in sensory learning. Sensory learning in this context refers to the application of sensory feedback on a patient's residual limb. Sensory feedback has been found to increase a user's willingness to use a prosthesis and leads to more active use.

Recent advances in prosthetic systems incorporate biofeedback that allows the user to “feel” through the prosthesis system. Specifically, sensors implanted on a user's residual limb can be used to provide physiologically appropriate sensory information to stimulate a patient's median and ulnar nerves enabling them to modulate the prosthesis without visual or auditory clues. Generally, these prosthesis systems either integrate sensors on the socket wall, insert sensors inside the socket, or embed sensors into the socket wall. Inserting sensors inside the socket requires extra thin, yet durable technology which restricts the number of options. Consequently, these systems tend to have poor accuracy and sensitivity. Integrating sensor systems on and into the socket require extensive work by a skilled prosthetist that is expensive and time consuming. Finally, prosthetic liners with sensor systems must be custom made to a user's requirements to avoid discomfort which requires a labor-intensive and expensive process.

Some prosthesis systems utilize microprocessors to interpret and analyze signals from angle and moment sensors. The microprocessors use these signals to determine the type of motion the patient is engaged in and modulates joints in the prosthesis accordingly, by varying the resistance to regulate the extension and compression of the prosthesis.

Prosthetic users frequently experience skin problems due to functional loss, prosthetic fitting and alignment issues, and amputation-associated medical problems such as skin disorders that are secondary to the use of the artificial limb. Skin lesions, and other extensive skin disorders, may start as an irritation and grow to become a potentially dangerous symptom, particularly in the diabetic population. The effects of pressure and friction on an amputee's skin can cause a myriad of skin disorders, from contact dermatitis to verrucose hyperplasia and various bacterial and fungal infections.

### BRIEF DESCRIPTION OF THE FIGURES

The various advantages of the embodiments will become apparent to one skilled in the art by reading the following specification and referencing the following figures, in which:

FIG. 1 is a high-level flow diagram comprising the steps of the tissue-prosthesis interface sensor system according to an embodiment.

FIG. 2 is a low-level flow diagram showing an embodiment of the tissue-prosthesis interface sensor system comprising a network of high baud rate, real-time, quantitative feedback multiplexers used to control additional multiplexers, and in turn interface with sensors and LEDs.

FIG. 3 is an embodiment of the disclosed tissue-prosthesis interface sensor system comprising two multiplexer layers and a microcontroller, wherein the microcontroller control pins select which multiplexer and which input/output pins on a multiplexer it reads from.

### DETAILED DESCRIPTION OF THE ART

The disclosed invention comprises a tissue-prosthesis interface sensor system used to measure pressure values exerted across the surface of the limb. The tissue-prosthesis interface sensor system provides a high-speed method of measuring pressure values through sensors located on the surface of a wearer's skin. This tissue-prosthesis interface sensor system may also work with the technology described in U.S. patent application Ser. No. 17/308,737 by incorporating sensors into some or all of the modular linkages throughout the tissue-prosthesis interface sensor system, either between the skin and silicone coverings or between the silicone coverings modules.

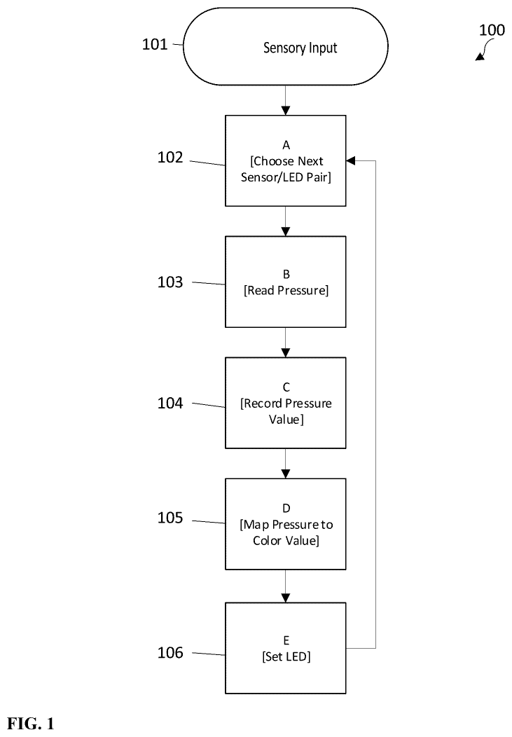

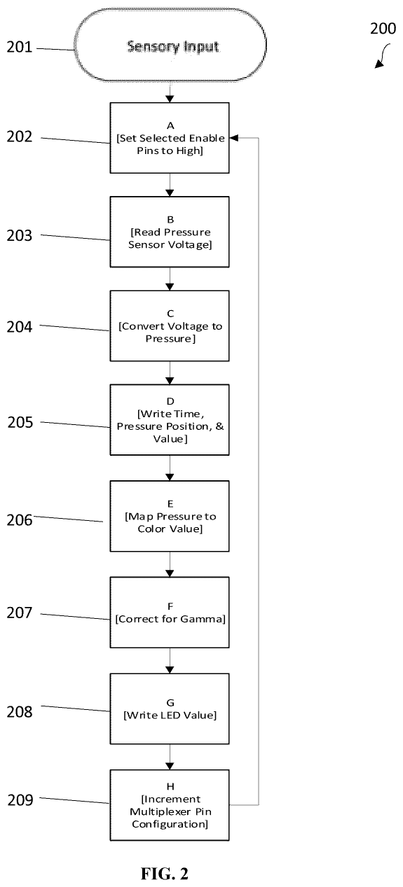

FIGS. 1 and 2 illustrate the algorithm that enables the tissue-prosthesis interface sensor system to read data in real time, according to an embodiment. From a high-level perspective, the microcontroller (described in detail below) first selects a sensor to read an input value from, then reads and records the input value, and maps the input value to an output value. An embodiment of the tissue-prosthesis interface sensor system may utilize NeoPixel digital RGB LEDs to visually indicate the pressure load detected. For example, in this embodiment, green might indicate a pressure load within acceptable limits and red might indicate a pressure load that exceeds safe limits. An embodiment may also utilize flashing LEDs to signal when an injurious load is detected. This process then repeats until all sensors are read and outputs written. The cycle loops to provide continuous, real-time measurements.

Referring to FIG. 1, at 101 a sensory input occurs. At 102, a particular sensor/LED pair is chosen. At 103, the pressure reading for the particular sensor is read. At 104, the pressure reading from 103 is recorded. At 105, the pressure reading from 103 is mapped to a color value. At 106, an LED is set to the corresponding color value determined in 105.

Referring to FIG. 2, this shows a more detailed version of the process of FIG. 1, in an embodiment. At 201, a sensory input occurs. At 202, the pins to a corresponding multiplexer output are set to high. At 203, the pressure is read as the sensor's voltage. At 204, the voltage from 203 is converted as a pressure. At 205, the time and position of the sensor which read the pressure are recorded. At 206, the pressure is mapped to a color value. At 207, the color mapped in 206 is then corrected for gamma using methods known to somebody of skill in the art. At 208, the LED is set to the color mapped in 206, incorporating the correction in 207. At 209, the system increments to the next multiplexer pin.

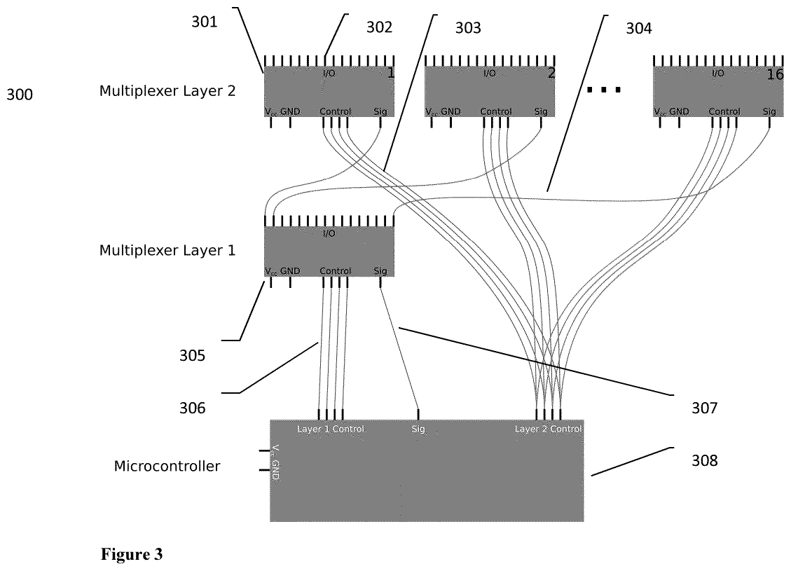

FIG. 3 shows an embodiment of the sensor system's circuitry. The tissue-prosthesis interface sensor system comprises a plurality of interconnected multiplexer layers and one or more microcontrollers. In the highest layer (i.e. multiplexer layer 2 of FIG. 3), each multiplexer comprises a plurality of input/output pins which each interface with a sensor that can read data (e.g. pressure or temperature values) from the user's skin. In the embodiment shown in FIG. 3, there are 16 pins on each multiplexer. The multiplexers in the layer below (multiplexer layer 1, FIG. 3) also comprise input/output pins that interface with the signal pin on the multiplexers in the layer above.

Multiplexers are layered to multiply the number of available pins and thus sensors which can be read from (see FIG. 3). The signal pins 304 on each multiplexer within a layer connect to an input/output pin on a multiplexer in the layer below. The signal pin 307 on the lowest layer (layer 1 in FIG. 3) 305 connects to the signal pin of a microcontroller 308. Thus, a signal pin 302 on the multiplexer layer (layer 2) 301 connected to the sensors obtains the recorded data value and transmits it to the layer below (layer 1) 304 and ultimately to the microcontroller 307. The microcontroller then transmits the value to an output, such as an LED to visually indicate the data value, e.g. a pressure load. To reiterate, layer 2 reads in information from the sensors. A layer 2 multiplexer transmits the data through its signal pins and layer 1 reads in this data through its input/output pins. Layer 1 then transmits this information to the microcontroller through the connection between the respective signal pins on the multiplexer in layer 1 and microcontroller.

The multiplexers and microcontroller further comprise a plurality of control pins. The control pins allow multiplexers to connect to a common set of control pins on the microcontroller 306, henceforth “layer 2 control pins.” The stacked arrangement of multiplexer layers and these control pins allow layer 2 control pins on the microcontroller to modulate both the input multiplexer(s) pins 303 as well as the output multiplexer(s) pins. In doing so, the microcontroller controls which multiplexer and input/output pin is read. Layer 1 control pins 306 on the microcontroller 308 modulate which multiplexer in layer 2 is read from. The layer 2 control pins 303 on the microcontroller 308 modulate which pins on a multiplexer in layer 2 are read. As a result, the tissue-prosthesis interface sensor system is able to provide the wearer and clinicians with real-time quantitative feedback at a high baud rate (i.e. the tissue-prosthesis interface sensor system can absorb, filter, and process large amounts of data very quickly).

In the embodiment illustrated in FIG. 3, there is one multiplexer in layer 1 with 16 input/output pins. The number of multiplexers in layer 2 is only limited by the number of pins in the layer below and so in this embodiment, layer 2 could have up to 16 multiplexers. Thus, the microcontroller in this embodiment can modulate input from 256 (16 pins×16 multiplexers) pins, and thus 256 sensors.

The disclosed tissue-prosthesis interface sensor system which incorporates a stacked arrangement of multiplexers operates by multiplying identical input and output pin configurations. The tissue-prosthesis interface sensor system eliminates multiple switching times and increases the speed at which data is read or written. These benefits permit clinicians to identify socket-limb shape mismatch, identify regions of heightened or dulled sensitivity on the limb, assess and diagnose nerve or other sensory or circulatory disorders, and if used as a research tool, to quantifiably measure tissue-prosthesis interface sensor system load distribution.

The tissue-prosthesis interface sensor system provides real-time results through visual indicators and may also collect data for later diagnostic analysis. In a clinical setting, an embodiment of the disclosed invention might be used to monitor and change a patient's pressure load. For example, the tissue-prosthesis interface sensor system might be used on patients with diabetes who lose sensitivity in affected limbs. The pressure load diagnostics may be used to prevent injury from otherwise undetected loads or pressure loads that occur in one region for an extended period of time. The visual indicators are set to a specific color depending on whether a triggering event occurs. In one variation, the color of a specific LED may turn from green to red when an injurious load is detected. The injurious load which would cause a triggering event would be determined by comparing the load at a particular sensor to the maximum safe load. If the sensor's load is greater than the maximum safe load the system would consider this a triggering event. If the sensor's load is less than the maximum safe load, no triggering event occurs, and the microcontroller can iterate to the next sensor. Another variation may be that a LED emits light when a triggering event occurs and is otherwise off.

Information fed to the microcontroller can also be used to improve the tissue-prosthesis interface sensor system in the future. The continuous feed of data can be used to improve the user's experience by giving insightful feedback about patterns of the prosthetics operation. Running a machine learning algorithm on the data can be useful for analytical purposes. The information can be used to identify the method or locations most and least prone to injury or in need of readjustment. The data can then present this data to the user for a better experience in the future.

The invention can be applied to circumstances beyond the exemplary embodiments above. The use of a configuration of sensors feeding data to stacked multiplexers and a microcontroller as a means of accessing a large plurality of sensor signals in a deliberate and controlled sequence. For example, this architecture can be used in reading and monitoring a large number of security sensors, or in the real time monitoring of a large set of parameters as in the case of operating a motor vehicle. The sensors can be used to detect any number of physical phenomena separately or in conjunction with one another.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. [27] The breadth and scope of the present invention should not be limited by any of the above-described exemplary embodiments, but should be defined only in accordance with the following claims and their equivalents.

What is being claimed:

1. A prosthetic limb for detecting injurious conditions comprising:
a series of sensors throughout a cavity of the prosthetic limb for continuously detecting a particular physical condition, at each of the respective locations of the sensors, of the prosthetic limb;
a series of multiplexers configured to receive signals from the sensors and output data indicative of the physical condition detected at a particular sensor;
a microcontroller programmed to receive the data output by the series multiplexers and to compute and output a signal indicating that the condition of the prosthetic limb is either safe or unsafe at the particular sensor;
means of notifying the user of the physical condition within the prosthetic limb; and
a power source electrically connected to the sensors and the microcontroller.

2. The prosthetic limb of claim 1, wherein the series of multiplexers comprises one or more layers.

3. The prosthetic limb of claim 2, wherein the microcontroller is configured to receive and process data output from a lowest layer of the series of multiplexers.

4. The prosthetic limb of claim 1, wherein the means of notifying the user of the physical condition performs the notification in real time.

5. The prosthetic limb of claim 4, wherein the notification informs the user that adjustment of the prosthetic limb is necessary.

6. The prosthetic limb of claim 1, wherein the power source comprises a grounded source or one or more batteries.

7. A tissue-prosthesis interface sensor system for continuously monitoring a physical condition of a cavity of a prosthetic limb to ensure that injurious conditions are promptly corrected comprising;
a series of sensors throughout the cavity of the prosthetic limb configured to continuously measure a particular physical condition within the cavity;
at least one layer of multiplexers that receives input signals from the sensors and converts the signal into a value which is then output;
a microcontroller which receives the value output from the at least one layer of multiplexers, compares the value with predetermined values which are known to be safe, and outputs an indication as to whether the prosthetic limb is safely attached or is potentially injurious; and
a means of conveying to a user that the prosthetic limb is currently attached safely or is potentially injurious, based on the indication output by the microcontroller.

8. The system of claim 7, wherein the sensors are configured to continuously output the particular physical condition at respective locations, to the at least one layer of multiplexers.

9. The system of claim 7 wherein the at least one layer of multiplexers comprises means for sequentially directing each sensor's output of the particular physical condition at the sensor, to the microcontroller.

10. A method of improving system use comprising:
training the system using training data collected from regular use of a prosthetic limb;
determining one or more patterns about usage of said prosthetic limb;
notifying a user of the patterns pertaining to the usage of the prosthetic limb and implications of the patterns.

11. A system for detecting a triggering event comprising:
a series of sensors continuously detecting either a particular physical condition or various physical conditions, at each of the respective locations of the sensors;
a series of multiplexers configured to receive signals from the sensors and output data indicative of a particular physical condition detected at a particular one of the sensors;
a microcontroller programmed to receive the data output by the series of multiplexers and to compute and output a signal indicating the particular physical condition;
means of notifying the user of the particular physical condition; and
a power source electrically connected to the sensors and the microcontroller.
